(19) United States
(12) Patent Application Publication
(10) Pub. No.: US 2024/0226226 A1
(43) Pub. Date: Jul. 11, 2024

(54) KAPPA-OPIOID RECEPTOR AGONIST IMPLANTS FOR TREATMENT OF PRURITUS

(71) Applicant: Titan Pharmaceuticals, Inc., South San Francisco, CA (US)

(72) Inventors: Katherine BEEBE DEVARNEY, Aptos, CA (US); Marc RUBIN, Durham, NC (US); Rajesh A. PATEL, Redwood City, CA (US); Sunil SREEDHARAN, South San Francisco, CA (US)

(21) Appl. No.: 18/554,731

(22) PCT Filed: Apr. 14, 2022
(86) PCT No.: PCT/US2022/071728

§ 371 Date: Oct. 10, 2023

Related U.S. Application Data
(60) Provisional application No. 63/174,958, filed on Apr. 14, 2021.
(60) Provisional application No. 63/213,485, filed on Jun. 22, 2021.

(51) Int. Cl. A61K 38/07; A61K 9/00; A61K 45/06; A61K 47/32; A61P 17/04

(52) U.S. Cl. CPC A61K 38/07; A61K 9/0024; A61K 45/06; A61K 47/32; A61P 17/04

(57) ABSTRACT

The present disclosure provides compositions, methods and kits for treatment of pruritus (itch) in an individual. The compositions, methods, and kits are for administering an implant comprising a kappa-opioid receptor agonist to an individual, where the implant comprises a sustained release composition comprising a kappa-opioid receptor agonist and a biocompatible polymeric matrix. Implantation of the device allows a controlled release of kappa-opioid receptor agonist for an extended period of time. The implant may be implanted subcutaneously in an individual in need of continuous treatment with a kappa-opioid receptor agonist for treatment and relief of pruritus.

### CROSS REFERENCE TO RELATED APPLICATIONS

This application claims priority benefit of U.S. Provisional Patent Application No. 63/174,958 filed Apr. 14, 2021, and of U.S. Provisional Patent Application No. 63/213,485 filed Jun. 22, 2021. The entire contents of those applications are hereby incorporated by reference herein.

### TECHNICAL FIELD

The disclosure provides implants for long-term treatment of pruritus, comprising a biocompatible polymer matrix, a kappa-opioid receptor agonist (which can be a peptide), and optionally additional therapeutic agents. The biocompatible polymer matrix can be a homogenous matrix, or may comprise a core polymeric material and one or more additional layers.

### BACKGROUND OF THE INVENTION

Pruritus, or itchy skin, is a common dermatological complaint, which can be caused by many local or systemic conditions. Mild cases of pruritus can be treated with over-the-counter remedies, such as moisturizing creams or lotions, corticosteroid creams, and similar topical treatments, or with topical or oral anti-histamines.

Severe or chronic pruritus, or pruritus that cannot be controlled with over-the-counter remedies, requires more intensive treatment. Anti-depressants such as fluoxetine and phototherapy have been used to treat severe or chronic itching. However, these treatments often fail to provide relief. In particular, uremic pruritus (chronic kidney disease-associated pruritus or CKD-aP) is reported by nearly half of patients undergoing hemodialysis, is often refractory to treatment, and can last for months or years.

Accordingly, new therapies are needed for treatment of pruritus, such as uremic pruritus.

F. M. Hughes, Jr. et al. have reported on short peptides which are potent agonists of the kappa-opioid receptor (Open Med Chem J. 2013; 7:16-22), and proposed using the peptide for pain control. International Patent Application WO 2017/210668 is directed to sustained release compositions of kappa-opioid receptor agonists, including compounds described in Hughes et al.

The current disclosure provides for long-term treatment of pruritus using kappa-opioid receptor agonists, including kappa-opioid receptor agonist peptides and derivatives of kappa-opioid receptor agonist peptides, which are administered in implants.

### BRIEF SUMMARY OF THE INVENTION

The present disclosure provides implants (i.e., implantable drug delivery devices) comprising kappa-opioid receptor agonists, such as kappa-opioid receptor agonist peptides. The present disclosure also provides methods of treating pruritus using such implants.

In some embodiments, the concentration of kappa-opioid receptor agonist in each layer of the implant is designed such that an approximately constant or essentially constant amount of kappa-opioid receptor agonist is released from the implant over a period of time. The period of time may be at least about 1 month, at least about 2 months, at least about 3 months, at least about 6 months, at least about 9 months, at least about 12 months, at least about 15 months, at least about 18 months, at least about 21 months, or at least about 24 months.

In some embodiments, the disclosure provides a method of treating pruritus in an individual, comprising administering an implant comprising a sustained release composition to the individual, wherein the sustained release composition comprises a kappa-opioid receptor agonist and a biocompatible polymeric matrix, wherein the sustained release composition is configured to release a therapeutically effective amount of the kappa-opioid receptor agonist after administration. The kappa-opioid receptor agonist can comprise a peptide. The kappa-opioid receptor agonist can comprise a compound of formula I:

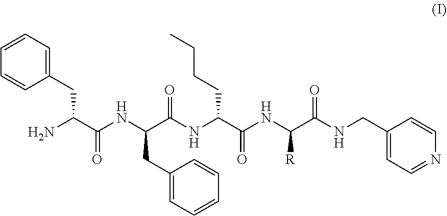

or a pharmaceutically acceptable salt or stereoisomer thereof, wherein R is

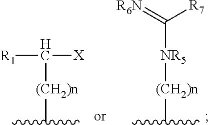

wherein n is an integer from 1 to 4; X is —NR₂R₃ or —N^{⊕}R₂R₃R₄; each of R₁, R₂, R₃, and R₄ is independently hydrogen, C₁-C₅ alkyl, C₁-C₅ substituted alkyl, C₂-C₅ alkenyl, C₂-C₅ substituted alkenyl, C₂-C₅ alkynyl, C₂-C₅ substituted alkynyl, C₃-C₆ cycloalkyl, C₆-C₁₀ aryl, C₆-C₁₀ substituted aryl, or —C₁-C₅ alkyl-C₆-C₁₀ aryl; R₇ is hydrogen, C₁-C₅ alkyl, C₁-C₅ substituted alkyl, C₂-C₅ alkenyl, C₂-C₅ substituted alkenyl, C₂-C₅ alkynyl, C₂-C₅ substituted alkynyl, C₃-C₆ cycloalkyl, C₆-C₁₀ aryl, C₆-C₁₀ substituted aryl, —C₁-C₅ alkyl-C₆-C₁₀ aryl, or —NR₈R₉; each of R₅, R₆, R₈, and R₉ is independently hydrogen, C₁-C₅ alkyl, C₁-C₅ substituted alkyl, C₁-C₅ alkenyl, C₁-C₅ substituted alkenyl, C₁-C₅ alkynyl, C₁-C₅ substituted alkynyl, C₃-C₆ cycloalkyl, C₆-C₁₀ aryl, C₆-C₁₀ substituted aryl, or —C₁-C₅ alkyl-C₆-C₁₀ aryl; or alternatively, R₈ and R₉ taken together with the nitrogen atoms to which they are attached form a heterocyclic ring; or alternatively, R₆ and R₉ taken together with the nitrogen atoms to which they are attached form a heterocyclic ring.

In some embodiments of Formula I, R is:

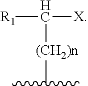

In some embodiments of Formula I, R is:

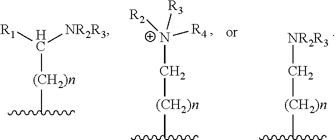

In some embodiments of Formula I, R is:

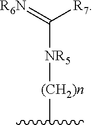

In some embodiments of Formula I, R is:

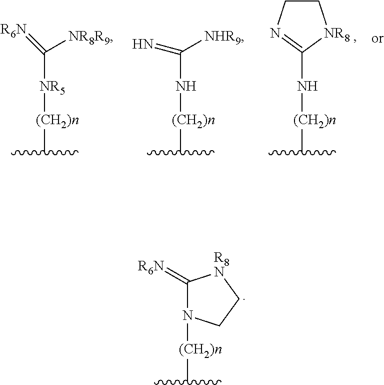

In some embodiments of Formula I, R is:

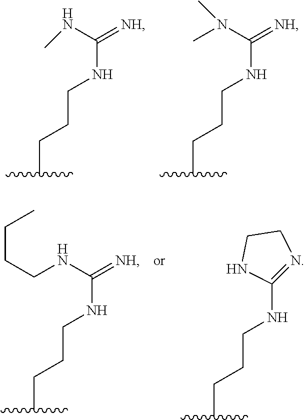

In some embodiments of Formula I, R is:

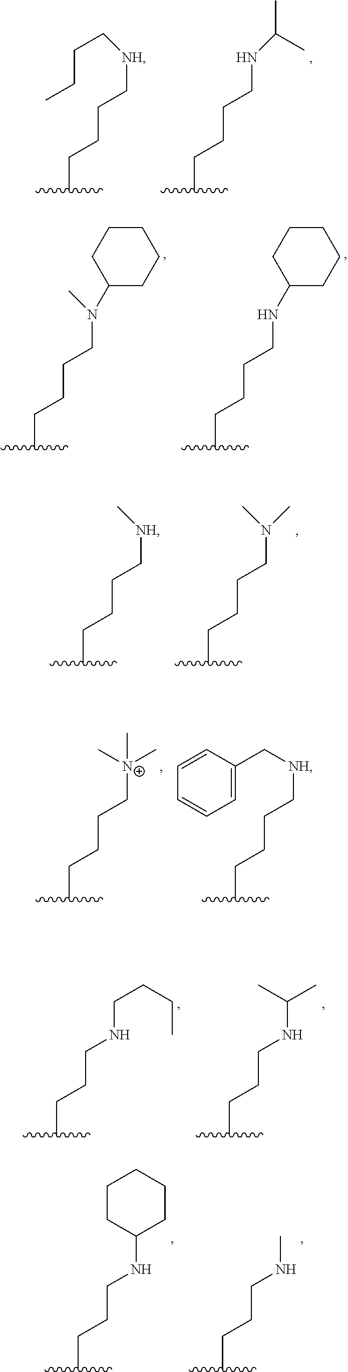

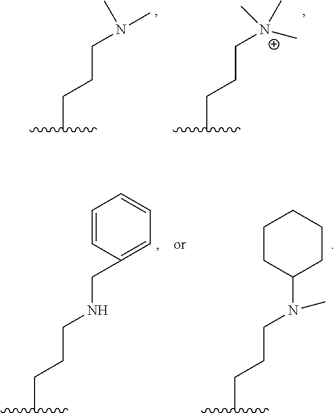

In some embodiments of Formula I, R is:

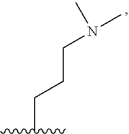 [SMILES: *CCCN(C)C]

and the compound of Formula (I) is Compound A:

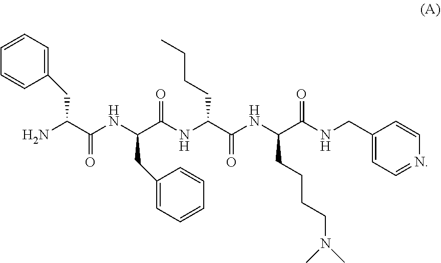 [SMILES: CCCC[C@@H](NC(=O)[C@@H](Cc1ccccc1)NC(=O)[C@H](N)Cc1ccccc1)C(=O)N[C@H](CCCCN(C)C)C(=O)NCc1ccncc1]

In any of the foregoing embodiments, the biocompatible polymeric matrix can comprise ethylene vinyl acetate (EVA) copolymer, crosslinked poly(vinyl alcohol), poly(hydroxy ethylmethacrylate), an acyl substituted cellulose acetate, a hydrolyzed alkylene-vinyl acetate copolymer, polyvinyl chloride, polyvinyl acetate, a polyvinyl alkyl ether, polyvinyl fluoride, polycarbonate, polyurethane, polyamide, polysulphone, styrene acrylonitrile copolymer, crosslinked poly(ethylene oxide), a poly(alkylene), poly(vinyl imidazole), a poly(ester), poly(ethylene terephthalate), polyphosphazene, a chlorosulphonated polyolefin, poly-lactide (PLA), poly-glycolide (PGA), poly(lactic-co-glycolic acid) (PLGA), or combinations thereof.

In any of the foregoing embodiments, the biocompatible polymeric matrix can comprise ethylene vinyl acetate (EVA) copolymer. In some embodiments, the EVA copolymer can comprise about 20% to about 40% vinyl acetate of the total weight of the copolymer. In some embodiments, the EVA copolymer can comprise about 33% vinyl acetate of the total weight of the copolymer.

In any of the foregoing embodiments, the kappa-opioid receptor agonist comprises about 10% to about 85% of the total weight of the sustained release composition, such as about 30% about 70% of the total weight of the sustained release composition.

In any of the foregoing embodiments, the implant can be a rod-shaped device having a diameter of about 0.5 mm to about 10 mm, and a length of about 0.5 cm to about 10 cm.

In any of the foregoing embodiments, the implant can be a rod-shaped device having a diameter of about 2 mm to about 3 mm, and a length of about 2 cm to about 4 cm.

In any of the foregoing embodiments, the implant can release about 0.1 mg to about 10 mg of the kappa-opioid receptor agonist per day.

In any of the foregoing embodiments, the implant can be implanted subcutaneously.

In any of the foregoing embodiments, the implant can comprise a core comprising the sustained release composition and a first layer comprising a first-layer biocompatible polymeric matrix surrounding the core.

In any of the foregoing embodiments having a first layer, the first-layer biocompatible polymeric matrix can comprise ethylene vinyl acetate (EVA) copolymer, crosslinked poly(vinyl alcohol), poly(hydroxy ethylmethacrylate), an acyl substituted cellulose acetate, a hydrolyzed alkylene-vinyl acetate copolymer, polyvinyl chloride, polyvinyl acetate, a polyvinyl alkyl ether, polyvinyl fluoride, polycarbonate, polyurethane, polyamide, polysulphone, styrene acrylonitrile copolymer, crosslinked poly(ethylene oxide), a poly(alkylene), poly(vinyl imidazole), a poly(ester), poly(ethylene terephthalate), polyphosphazene, a chlorosulphonated polyolefin, poly-lactide (PLA), poly-glycolide (PGA), poly(lactic-co-glycolic acid) (PLGA), or combinations thereof.

In any of the foregoing embodiments having a first layer, the first-layer biocompatible polymeric matrix can comprise ethylene vinyl acetate (EVA) copolymer. The EVA copolymer of the first-layer biocompatible polymeric matrix can comprise about 20% to about 40% vinyl acetate of the total weight of the copolymer. The EVA copolymer of the first-layer biocompatible polymeric matrix can comprise about 33% vinyl acetate of the total weight of the copolymer.

In any of the foregoing embodiments, the implant can further comprise one or more additional layers comprising a biocompatible polymeric matrix.

In any of the foregoing embodiments having a core, a first layer, and optionally one or more additional layers, any of the core, the first layer, and the one or more additional layers if present, further comprise one or more additional pharmaceutical substances.

In any of the foregoing embodiments, the implant can remain in the individual for at least about 3 months, at least about 6 months, at least about 9 months, at least about 12 months, at least about 15 months, at least about 18 months, at least about 21 months, or at least about 24 months.

In any of the foregoing embodiments, the concentration of pharmaceutical substance in the blood plasma can be approximately constant or essentially constant for at least about 3 months, at least about 6 months, at least about 9 months, at least about 12 months, at least about 15 months, at least about 18 months, at least about 21 months or at least about 24 months.

In any of the foregoing embodiments, the kappa-opioid receptor agonist can be a compound of Formula (I).

In any of the foregoing embodiments, the kappa-opioid receptor agonist can be Compound A.

### BRIEF DESCRIPTION OF THE DRAWINGS

FIG. 1 shows plasma levels of kappa-opioid receptor agonist in rats having an implant with a core comprised of 50% of the kappa-opioid receptor agonist and 50% ethylene vinyl acetate (EVA), and a shell of 3% of the kappa-opioid receptor agonist and 97% EVA surrounding the core.

FIG. 2 shows scratching events in mice after administration of 5′-guanidinonaltrindole (5′-GNTI). The dashed line represents scratching events in mice (N=7) with a placebo implant or sham surgery, while the solid line represents scratching events in mice (N=5) with an implant comprising Compound A.

FIG. 3 shows plasma levels over time in mice resulting from an implant comprising Compound A (solid line), versus in mice resulting from a subcutaneous injection of Compound A (dashed line).

FIG. 4 shows simulated human plasma levels for implants containing Compound A, versus a bolus injection of CR-845.

FIG. 5 shows reduced scratch events in mice with Compound A-containing implants, versus control mice.

### DETAILED DESCRIPTION OF THE INVENTION

The disclosure provides compositions (i.e., implants), methods, and kits for providing kappa-opioid receptor agonists to an individual.

“Drug” and “pharmaceutical substance” are equivalent terms and are used interchangeably.

In one aspect, the implant comprises a sustained release composition comprising a biocompatible polymeric matrix and a kappa-opioid receptor agonist, for example, up to approximately 5%, up to approximately 10%, up to approximately 20%, up to approximately 25%, up to approximately 30%, up to approximately 40%, up to approximately 50%, up to approximately 60%, up to approximately 70%, up to approximately 80%, or up to approximately 85% of kappa-opioid receptor agonist, with the remainder of the sustained release composition comprising the biocompatible polymeric matrix.

In some embodiments, the implant comprises a core comprising a biocompatible polymeric matrix and a kappa-opioid receptor agonist, and one or more additional layers surrounding the core. In some embodiments, the core is rod-like or cylindrical. In some embodiments, the core is rod-like or cylindrical, and is rounded at either end, that is, capped by a hemisphere, oblate hemisphere, oblate hemispheroid, or ellipsoid having about the same diameter as the rod-like or cylindrical portion of the core. The portions of the implant capping the ends of the rod can be essentially 100% polymer, or can contain the same percentage of polymer and kappa-opioid receptor agonist as in the core of the implant, or can comprises about or at least about 50% polymer and about or at most about 50% kappa-opioid receptor agonist, about or at least about 60% polymer and about or at most about 40% kappa-opioid receptor agonist, about or at least about 70% polymer and about or at most about 30% kappa-opioid receptor agonist, about or at least about 75% polymer and about or at most about 25% kappa-opioid receptor agonist, about or at least about 80% polymer and about or at most about 20% kappa-opioid receptor agonist, about or at least about 85% polymer and about or at most about 15% kappa-opioid receptor agonist, about or at least about 90% polymer and about or at most about 10% kappa-opioid receptor agonist, about or at least about 95% polymer and about or at most about 5% kappa-opioid receptor agonist, about or at least about 96% polymer and about or at most about 4% kappa-opioid receptor agonist, about or at least about 97% polymer and about or at most about 3% kappa-opioid receptor agonist, about or at least about 98% polymer and about or at most about 2% kappa-opioid receptor agonist, or about or at least about 99% polymer and about or at most about 1% kappa-opioid receptor agonist. In some embodiments the core extends the majority of the length of the implant. In some embodiments, the core comprises a lower concentration of kappa-opioid receptor agonist than the layer or layers surrounding it. In some embodiments, the core comprises a higher concentration of kappa-opioid receptor agonist than the layer or layers surrounding it. In some embodiments, the core comprises a kappa-opioid receptor agonist and is surrounded by a layer which does not comprise kappa-opioid receptor agonist.

In some aspects, wherein the implant comprises EVA, the vinyl acetate content is about 33% by weight. The implants generally comprise about 10% to about 85% kappa-opioid receptor agonist, such as about 10% to about 25% kappa-opioid receptor agonist, about 20% to about 40% kappa-opioid receptor agonist, about 25% to about 75% kappa-opioid receptor agonist, about 30% to about 70% kappa-opioid receptor agonist, or about 40% to about 60% kappa-opioid receptor agonist. In one embodiment, the implant comprises about 50% kappa-opioid receptor agonist.

Another aspect of this disclosure is a method for delivering a kappa-opioid receptor agonist to an individual in need thereof, comprising the step of inserting an implant subcutaneously into the individual, wherein the kappa-opioid receptor agonist is released from the implant into the individual. In some embodiments, the individual in need thereof is suffering from pruritus. In some embodiments, the individual in need thereof is suffering from uremic pruritus.

In some embodiments, the individual is a human. In some embodiments, the individual is a mammal, such as a primate including chimpanzees, orangutans, gorillas, and monkeys; a domestic animal or pet including dogs, cats, and guinea pigs; a laboratory animal such as a rat or a mouse; or an animal used in agriculture such as a cow, sheep, or pig.

In one aspect, the implant remains implanted in the individual for at least about 1 month, at least about 2 months, at least about 3 months, at least about 6 months, at least about 9 months, at least about 12 months, at least about 15 months, at least about 18 months, at least about 21 months, or at least about 24 months.

In another aspect, the concentration of kappa-opioid receptor agonist in each layer of the implant is designed such that a steady-state level or approximately constant level or essentially constant level of kappa-opioid receptor agonist is released into the individual. In another aspect, the implants provide a steady-state level or approximately constant level or essentially constant level of kappa-opioid receptor agonist in the plasma of the individual.

In any of the embodiments disclosed herein, the core can comprise a polymer, for example ethylene vinyl acetate, mixed with a radiopaque compound. In some embodiments, the radiopaque compound is barium sulfate. In some embodiments, the radiopaque compound is bismuth subcarbonate, bismuth trioxide, or tungsten. Inclusion of a radiopaque compound permits localization and visualization of the implant in-vivo via X-ray or ultrasound imaging.

In one embodiment, the disclosure provides a rod-shaped core comprising a polymer with essentially no pharmaceutical substance. This core is surrounded by a single layer comprising a polymer and a kappa-opioid receptor agonist. In one embodiment of this type, the rod is about 2 to about 3 cm in length, e.g., about 2.6 cm, and about 2 mm to about 3 mm in diameter; the single layer is about 0.5 mm to about 1 mm in thickness, and the core is about 0.5 mm to about 2 mm in diameter. In one embodiment, both the core and the single layer comprise the same polymer, for example, ethylene vinyl acetate (EVA). In some embodiments, the core comprises a polymer, for example, ethylene vinyl acetate (EVA); the layer comprises a different polymer, e.g., a bioerodible polymer such as PLGA. The single layer comprises about 10% to about 85% of a kappa-opioid receptor agonist.

In one embodiment, the disclosure provides a rod-shaped core comprising a polymer with essentially no pharmaceutical substance. This core is surrounded by two layers comprising a polymer and a pharmaceutical substance, wherein at least one of the layers comprises a kappa-opioid receptor agonist. In one embodiment of this type, the rod is about 2 to about 3 cm in length, e.g., about 2.6 cm, and about 2 mm to about 5 mm in diameter; each layer is about 0.5 to about 1 mm in thickness, and the core is about 0.5 mm to about 2 mm in diameter. In one embodiment, the core and both layers all comprise the same polymer, for example, ethylene vinyl acetate (EVA). In some embodiments, the core comprises a polymer, for example, ethylene vinyl acetate (EVA); the layers comprise a different polymer, e.g., a bioerodible polymer such as PLGA. Both layers comprise a pharmaceutical substance, which may be the same substance in each layer or different substances in each layer, for example, a substance independently selected from additional kappa-opioid receptor agonists, mu-opioid receptor agonists, mu-opioid receptor antagonists, mixed mu-opioid receptor agonists/antagonists such as buprenorphine, delta-opioid receptor agonists, delta-opioid receptor antagonists, anti-inflammatory drugs, and steroids. At least one of the layers independently comprises about 10% to about 85% of kappa-opioid receptor agonist. In one embodiment, both layers contain the kappa-opioid receptor agonist, and the outermost layer comprises a lower concentration of the kappa-opioid receptor agonist than the innermost layer; e.g., the outermost layer comprises about 10% to about 90% of a kappa-opioid receptor agonist and the innermost layer comprises about 10% to about 90% of the kappa-opioid receptor agonist, where the outermost layer comprises a lower concentration of the kappa-opioid receptor agonist than the innermost layer.

In one embodiment, the disclosure provides a rod-shaped core comprising a polymer with essentially no pharmaceutical substance. The core is surrounded by three layers comprising a polymer and a pharmaceutical substance, wherein at least one of the layers comprises a kappa-opioid receptor agonist. In one embodiment of this type, the rod is about 2 to about 3 cm in length, e.g., about 2.6 cm, and about 3 mm to about 7 mm in diameter; each layer is about 0.5 mm to about 1 mm in thickness, and the core is about 0.5 to about 2 mm in diameter. In one embodiment, the core and all the layers all comprise the same polymer, for example, ethylene vinyl acetate (EVA). In some embodiments, the core comprises a polymer, for example, ethylene vinyl acetate (EVA); the layers comprise a different polymer, e.g., a bioerodible polymer such as PLGA. All the layers comprise a pharmaceutical substance which may be the same substance in each layer, different substances in each layer, or the same in two of the layers and different in the third layer, provided that at least one of the layers comprises a kappa-opioid receptor agonist. Other pharmaceutical substances which can be used in the layers include, for example, a substance independently selected from additional kappa-opioid receptor agonists, mu-opioid receptor agonists, mu-opioid receptor antagonists, mixed mu-opioid receptor agonists/antagonists such as buprenorphine, delta-opioid receptor agonists, delta-opioid receptor antagonists, anti-inflammatory drugs, and steroids. The layers can independently comprise about 10% to about 90% of the pharmaceutical substance(s). In one embodiment, each layer contains the same pharmaceutical substance, but the layers differ in the concentration of the pharmaceutical substance, such that the average concentration of the pharmaceutical substance in each layer decreases with increasing distance from the core. Thus the outermost layer comprises about 10% to about 90% of the pharmaceutical substance, while the middle layer comprises about 10% to about 90% of the pharmaceutical substance, and the innermost layer comprises about 10% to about 90% of the pharmaceutical substance, subject to the condition that the outer layer has a lower concentration of pharmaceutical substance than the middle layer, while the inner layer (adjacent to the core) has a higher concentration of pharmaceutical substance than the middle layer. In one such embodiment, the pharmaceutical substance in all three layers is a kappa-opioid receptor agonist.

In one embodiment, the disclosure provides a rod-shaped core comprising a polymer with essentially no pharmaceutical substance. The core is surrounded by four layers comprising a polymer and a pharmaceutical substance, wherein at least one of the layers comprises a kappa-opioid receptor agonist. In one embodiment of this type, the rod is about 2 cm to about 3 cm in length, e.g., about 2.6 cm, and about 4 mm to about 9 mm in diameter; each layer is about 0.5 mm to about 1 mm in thickness, and the core is about 0.5 mm to about 1 mm in thickness. In one embodiment, the core and all the layers all comprise the same polymer, for example, ethylene vinyl acetate (EVA). In some embodiments, the core comprises a polymer, for example, ethylene vinyl acetate (EVA); the layers comprise a different polymer, e.g., a bioerodible polymer such as PLGA. All the layers comprise a pharmaceutical substance, provided that at least one of the layers comprises a kappa-opioid receptor agonist. Other pharmaceutical substances which can be used in the layers include, for example, a substance selected from additional kappa-opioid receptor agonists, mu-opioid receptor agonists, mu-opioid receptor antagonists, mixed mu-opioid receptor agonists/antagonists such as buprenorphine, delta-opioid receptor agonists, delta-opioid receptor antagonists, anti-inflammatory drugs, and steroids. The layers can independently comprise about 10% to about 90% of the pharmaceutical substance(s). In one embodiment, each layer contains the same pharmaceutical substance, but the layers differ in the concentration of the pharmaceutical substance, such that the average concentration of the pharmaceutical substance in each layer decreases with increasing distance from the core. Thus the outermost layer comprises about 10% to about 90% of the pharmaceutical substance, the second-outermost layer comprises about 10% to about 90% of the pharmaceutical substance, the third-outermost layer comprises about 10% to about 90%, of the pharmaceutical substance and the innermost layer (adjacent to the core) comprises about 10% to about 90% of the pharmaceutical substance, subject to the condition that the outermost layer has a concentration of the pharmaceutical substance lower than the concentration in the second-outermost layer, the second-outermost layer has a concentration of the pharmaceutical substance lower than the concentration in the third-outermost layer, and the third-outermost layer has a concentration of the pharmaceutical substance lower than the innermost layer (adjacent to the core). In one such embodiment, the pharmaceutical substance in all four layers is a kappa-opioid receptor agonist.

In one embodiment, the disclosure provides a rod-shaped core comprising a polymer with essentially no pharmaceutical substance. The core is surrounded by five layers comprising a polymer and a pharmaceutical substance, wherein at least one of the layers comprises a kappa-opioid receptor agonist. In one embodiment of this type, the rod is about 2 cm to about 3 cm in length, e.g., about 2.6 cm, and about 5 mm to about 10 mm in diameter; each layer is about 0.5 mm to about 1 mm in thickness, and the core is about 0.5 mm to about 1 mm in thickness. In one embodiment, the core and all the layers all comprise the same polymer, for example, ethylene vinyl acetate (EVA). In some embodiments, the core comprises a polymer, for example, ethylene vinyl acetate (EVA); the layers comprise a different polymer, e.g., a bioerodible polymer such as PLGA. All the layers comprise a pharmaceutical substance, provided that at least one of the layers comprises a kappa-opioid receptor agonist. Other pharmaceutical substances which can be used in the layers include, for example, a substance independently selected from additional kappa-opioid receptor agonists, mu-opioid receptor agonists, mu-opioid receptor antagonists, mixed mu-opioid receptor agonists/antagonists such as buprenorphine, delta-opioid receptor agonists, delta-opioid receptor antagonists, anti-inflammatory drugs, and steroids. The layers can independently comprise about 10% to about 90% of the pharmaceutical substance(s). In one embodiment, each layer contains the same pharmaceutical substance, but the layers differ in the concentration of the pharmaceutical substance, such that the average concentration of the pharmaceutical substance in each layer decreases with increasing distance from the core. Thus the outermost layer comprises about 10% to about 90% of the pharmaceutical substance, the second-outermost layer comprises about 10% to about 90% of the pharmaceutical substance, the third-outermost layer comprises about 10% to about 90%, of the pharmaceutical substance and the innermost layer (adjacent to the core) comprises about 10% to about 90% of the pharmaceutical substance, subject to the condition that the outermost layer has a concentration of the pharmaceutical substance lower than the concentration in the second-outermost layer, the second-outermost layer has a concentration of the pharmaceutical substance lower than the concentration in the third-outermost layer, the third-outermost layer has a concentration of the pharmaceutical substance lower than the fourth-outermost layer, and the fourth-outermost layer has a concentration of the pharmaceutical substance lower than the innermost layer (adjacent to the core). In one such embodiment, the pharmaceutical substance in all five layers is a kappa-opioid receptor agonist.

In additional embodiments, the disclosed implants can comprise additional layers, each layer having a decreasing concentration of pharmaceutical substance as the distance from the core increases, in a manner similar to that described above.

In any of the above embodiments, one or more of the layers can be non-bioerodible. In any of the above embodiments, all of the layers can be non-bioerodible. In any of the above embodiments, the core can be non-bioerodible. In any of the above embodiments, the core and one or more of the layers can be non-bioerodible, with the proviso that no non-bioerodible material is external to a bioerodible layer or a bioerodible core (i.e., if the implant has any bioerodible layers, then any additional layers which are external to that layer must be bioerodible; equivalently, if the implant has any non-bioerodible layers, then any bioerodible layers are located external to that layer, that is, the bioerodible layers are farther from the core than any non-bioerodible layers. This condition also requires all layers to be bioerodible if the core is bioerodible). In any of the above embodiments, the core and all of the layers can be non-bioerodible.

In any of the above embodiments, one or more of the layers can be bioerodible. In any of the above embodiments, all of the layers can be bioerodible, while the core is non-bioerodible. In any of the above embodiments, the core and each of the one or more layers are bioerodible.

In any of the above embodiments, one or more of the layers can comprise a mixture of a bioerodible polymer and a non-bioerodible polymer. The mixture can be blended together prior to extruding in the same layer. Alternatively, the mixture can be coextruded into the same layer at the time of forming the layer. In various embodiments, the proportion of bioerodible polymer to non-erodible polymer in the mixed layer can be about 10% bioerodible and 90% non-erodible, about 20% bioerodible and 80% non-erodible, about 25% bioerodible and 75% non-erodible, about 30% bioerodible and 70% non-erodible, about 33% bioerodible and 67% non-erodible, about 40% bioerodible and 60% non-erodible, about 50% bioerodible and 50% non-erodible, about 60% bioerodible and 40% non-erodible, about 67% bioerodible and 33% non-erodible, about 70% bioerodible and 30% non-erodible, about 75% bioerodible and 25% non-erodible, about 80% bioerodible and 20% non-erodible, or about 90% bioerodible and 10% non-erodible. In one embodiment, the bioerodible polymer is chosen from any of the bioerodible polymers recited elsewhere in this specification. In one embodiment, the non-erodible polymer is chosen from any of the non-erodible polymers recited elsewhere in this specification. In any of the foregoing embodiments of mixed layers, the bioerodible polymer can be PLGA. In any of the foregoing embodiments of mixed layers, the non-erodible polymer can be EVA. In any of the foregoing embodiments of mixed layers, the bioerodible polymer can be PLGA and the non-erodible polymer can be EVA. When a layer is used which comprises a mixture of a bioerodible polymer and a non-bioerodible polymer, any layers external to that mixed layer are either bioerodible or mixed bioerodible/non-erodible.

In one embodiment, the disclosure provides a rod-shaped core comprising a polymer with essentially no pharmaceutical substance. This core is surrounded by two layers comprising a polymer and a pharmaceutical substance, provided that at least one layer comprises a kappa-opioid receptor agonist. In one embodiment of this type, the rod is about 2 cm to about 3 cm in length, e.g., about 2.6 cm, and about 2 mm to about 5 mm in diameter; each layer is about 0.5 mm to about 1 mm in thickness, and the core is about 0.5 mm to 2 mm in diameter. In one embodiment, the core and all the layers all comprise the same polymer, for example, ethylene vinyl acetate (EVA). In some embodiments, the core comprises a polymer, for example, ethylene vinyl acetate (EVA); the layers comprises a different polymer, e.g., a bioerodible polymer such as PLGA, and each layer comprises the same bioerodible polymer. Both layers comprise a pharmaceutical substance, provided that at least one of the layers comprises a kappa-opioid receptor agonist. Other pharmaceutical substances which can be used in the layers include, for example, a substance independently selected from additional kappa-opioid receptor agonists, mu-opioid receptor agonists, mu-opioid receptor antagonists, mixed mu-opioid receptor agonists/antagonists such as buprenorphine, delta-opioid receptor agonists, delta-opioid receptor antagonists, anti-inflammatory drugs, and steroids. In one embodiment, both layers contain the same pharmaceutical substance, and the outermost layer comprises a higher concentration of the pharmaceutical substance than the innermost layer; e.g., the outermost layer comprises about 10% to about 90% of a pharmaceutical substance and the innermost layer comprises about 10% to about 90% of the pharmaceutical substance, where the outermost layer comprises a higher concentration of the pharmaceutical substance than the innermost layer. In one such embodiment, the pharmaceutical substance in both layers is a kappa-opioid receptor agonist. Optionally, this embodiment can comprise additional layers comprising polymer and pharmaceutical substance; in this case, the outermost layer comprises the highest concentration of pharmaceutical substance, with each more inner layer comprising a lower level of drug. This configuration allows an initial high rate of drug release into the individual over a certain period, and thus a higher initial serum or systemic level of drug, followed by a decreasing release rate over time which results in a lower subsequent serum or systemic level of drug, which gradually decreases over a period of time. In some embodiments, wherein the outermost layer also comprises the highest concentration of drug, the inner-more layers can comprise approximately equal concentrations of drug, yet all lower than the outermost layer. This will allow an initial high rate of drug release into the individual over a certain period, and thus a higher initial serum or systemic level of drug, followed by a lower, essentially steady-state level of drug over a period of time.

In additional embodiments, the disclosed implant can comprise additional layers, each layer having an increasing concentration of pharmaceutical substance as the distance from the core increases.

In one embodiment, the disclosure provides a rod-shaped core comprising a polymer with essentially no pharmaceutical substance. This core is surrounded by a single layer comprising a polymer and a kappa-opioid receptor agonist. Finally, the single layer is surrounded by a layer of essentially pure pharmaceutical substance, which is optionally a kappa-opioid receptor agonist. In one embodiment of this type, the rod is about 2 to about 3 cm in length, e.g., about 2.6 cm, and about 2 mm to about 3 mm in diameter; the single layer is about 0.5 mm to about 1 mm in thickness, and the core is about 0.5 mm to about 2 mm in diameter, while the thickness of the layer of pure drug is determined by the amount of pure drug to be used. In one embodiment, both the core and the single layer comprise the same polymer, for example, ethylene vinyl acetate (EVA). In some embodiments, the core comprises a polymer, for example, ethylene vinyl acetate (EVA); the layer comprises a different polymer, e.g., a bioerodible polymer such as PLGA. The single layer comprises about 10% to about 90% of kappa-opioid receptor agonist. The layer of pure drug comprises about 100% of a pharmaceutical substance, independently selected from, for example, a kappa-opioid receptor agonist, a mu-opioid receptor agonist, a mu-opioid receptor antagonist, a mixed mu-opioid receptor agonist/antagonist such as buprenorphine, a delta-opioid receptor agonist, a delta-opioid receptor antagonist, an anti-inflammatory drug, and a steroid. In one embodiment, the pharmaceutical substance in both the single layer and the layer of pure drug is a kappa-opioid receptor agonist.

In additional embodiments, the disclosed implant can comprise additional layers, each layer having an increasing concentration of pharmaceutical substance as the distance from the core increases, with a layer of essentially pure pharmaceutical substance on the outside of the implant. This configuration allows an initial high rate of drug release into the individual over a certain period, and thus a higher initial serum or systemic level of drug.

In one aspect, the disclosure provides an implant for delivering a kappa-opioid receptor agonist, comprising the substance and a biocompatible polymeric matrix. The drug is encapsulated within the matrix, and the implant is subcutaneously implanted in a mammal such as a dog or cat or human being. The pharmaceutical substance is continuously released from the implant over a sustained period of time through pores that open in the surface of the matrix. After any initial burst of drug, the drug is delivered, for example, at a rate of at least about 0.1 mg per day, generally in the range of about 0.1 to about 5 mg per day. In some embodiments, the rate of drug release is about 0.01 mg per day, 0.02 mg per day, 0.03 mg per day, 0.04 mg per day, 0.05 mg per day, or 0.1 mg per day. In some embodiments, the rate of drug release is about 0.1 mg to about 0.5 mg per day, about 0.5 mg to about 1.0 mg per day, about 1.0 mg to about 3.0 mg per day, or about 3.0 mg to about 5.0 mg per day.

The rate of drug release, which is determined by the size and other physical parameters of the implant, implant location, and concentration of drug in various layers of the implant, can be tailored to provide a desired dosage in relation to an individual's ailment, physical condition and weight or body surface area.

### Kappa-Opioid Receptor Agonists

In any of the embodiments of the implants, sustained release compositions, or methods disclosed herein, the kappa-opioid receptor agonist can be a compound of Formula (I):

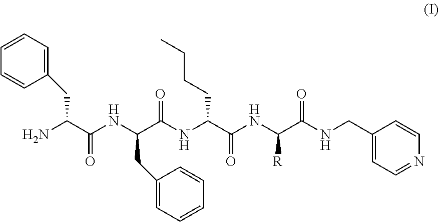

as defined herein.

In a preferred embodiment, in any of the embodiments of the implants, sustained release compositions, or methods disclosed herein, the kappa-opioid receptor agonist can be Compound A:

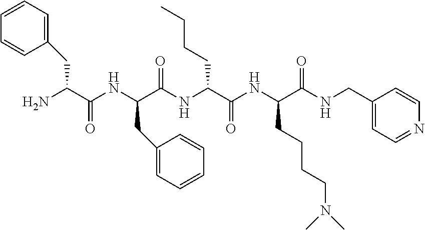 [SMILES: CCCC[C@@H](NC(=O)[C@@H](Cc1ccccc1)NC(=O)[C@H](N)Cc1ccccc1)C(=O)N[C@H](CCCCN(C)C)C(=O)NCc1ccncc1]

or a pharmaceutically acceptable salt or stereoisomer thereof.

### Manufacture of the Implants

In some embodiments, the implants can be produced by an extrusion process. The drug substance can be prepared by milling (e.g., ball-milling, impact-milling), spray-drying, solvent precipitation, screening or other method or combination of methods known in the art to produce fine particles. The drug can be combined with a polymer which is also prepared as fine particles. The blended mixture can be extruded, e.g., via Microtruder screw extruder, Model No. RCP-025, Randcastle Extrusion Systems, Cedar Grove, NJ, or via other extrusion devices known in the industry. The diameter of extrusion, as well as temperature, pressure and other parameters can be controlled as appropriate for each drug.

When EVA is used as the biocompatible polymeric matrix, implants may be produced using an extrusion process, wherein ground EVA is blended with kappa-opioid receptor agonist, melted, and extruded into rod-shaped structures. Rods are cut into individual implants of the desired length, packaged, and sterilized prior to use.

Other methods for encapsulating therapeutic compounds in implantable polymeric, nonerodible matrices are well known to those of skill in the art. Such methods include, for example, solvent casting (see, e.g., U.S. Pat. Nos. 4,883,666, 5,114,719, and 5,601,835). A skilled artisan would be able to readily determine an appropriate method of preparing such an implant, depending on the shape, size, kappa-opioid receptor agonist loading, and release kinetics desired for a particular type of individual or clinical indication.

In some embodiments, a core comprising polymer can be formed, e.g., by extrusion, which is then coated with one or more layers comprising polymer and drug via a dip coating or spray coating method. A solvent evaporation technique may be used to mix the polymer and drug in a solvent. The solution comprising polymer, drug and solvent can then be applied to the surface of the core by either dipping or spraying. The resultant composition is then subjected to a drying process, during which the solvent is evaporated, and the polymeric material, with the drug dispersed therein, forms a thin film or layer on the core. This procedure can be repeated with various solutions of the same or differing concentrations of drug and polymer to deposit additional layers on the composition. As is known in the art, implants comprising multiple layers may be produced by any combination of extrusion and coating.

The extrudate can be extruded horizontally and collected for further processing. The extrudate can be cut into desirable lengths, e.g., from about 1 to about 3 cm. The extrudate can then be washed in any solvent in which the drug or drugs dissolve, and then dried and packaged.

Examples of implantable polymeric devices for continuous drug release which can be used for delivery of kappa-opioid receptor agonists are described in, e.g., U.S. Pat. Nos. 4,883,666; 5,114,719; 5,601,835; 10,111,830; and 10,123,971; and Patel et al. U.S. Patent Application Publication No. 2021/0007973.

Implants with multiple layers can be produced by co-extrusion methods known in the art, for example, by the methods disclosed in U.S. Pat. No. 5,063,018 (for manufacturing catheters with a lumen), or U.S. Pat. Nos. 4,832,589, 4,379,117, 3,880,691, and 3,337,665. Multi-manifold dies, such as multi-manifold dies using feedblock co-extrusion, are known in the art for producing multi-layered materials.

The kappa opioid receptor agonists disclosed herein, such as Compound A, can be administered in heterogeneous implants, such as those disclosed in U.S. Pat. Nos. 10,111,830 and 10,123,971. Such an implant comprises a rod-shaped core comprising a core polymeric material, wherein the core does not comprise a pharmaceutical substance. The core polymeric material can be ethylene vinyl acetate (EVA). The core can have a diameter of about 0.5 mm to about 3.5 mm. The implant further comprises a first layer comprising a first-layer pharmaceutical substance surrounding the core. Optionally, the implant further comprises one or more additional layers, wherein each of the one or more additional layers comprise an independently selected additional pharmaceutical substance. The first layer can comprise EVA. The one or more additional layers can comprise EVA. The first layer can comprise a kappa opioid receptor agonist disclosed herein, such as Compound A, or any one or more of the one or more additional layers can comprise a kappa opioid receptor agonist disclosed herein, such as Compound A.

The kappa opioid receptor agonists disclosed herein, such as Compound A, can be administered in an implant having a core and a porogenic shell, such as the implants disclosed in U.S. Patent Application Publication No. US2021/0007973. Such an implant comprises a core comprising a first polymeric material and a core pharmaceutical substance; and a shell comprising a second polymeric material and a porogen material. The porogen material can be removed from the shell, leaving behind pores of defined size in the shell. The porogen material can comprise particles of between 1 micrometer and 300 micrometers in mean diameter, such as particles of between 1 micrometer and 50 micrometers in mean diameter. Porogen materials can be selected from an alkyl cellulose, a hydroxyalkyl cellulose, ethylcellulose, methylcellulose, hydroxymethylcellulose, a fatty acid, stearic acid, palmitic acid, myristic acid, linoleic acid, a biocompatible salt, sodium chloride, calcium chloride, sodium phosphate, benzoic acid, citric acid, polyvinylpyrrolidone (PVP). Salts of the foregoing acids can also be used, such as sodium or potassium salts.

### Physical Parameters of the Implants

In some embodiments, implants comprise dimensions of about 0.5 to about 7 mm in diameter. In some embodiments the implant is about 0.5 to 10 cm in length. In some embodiments, the implant is from about 1 to about 3 cm in length. In some embodiments, the implant is about 2 cm to about 3 cm in length. In some embodiments, the implant is about 2.6 cm in length. In some embodiments, the implant is about 1 to about 4 mm in diameter. In some embodiments, the implant is about 1 to about 3 mm in diameter. In some embodiments, the implant is about 2 to about 3 mm in diameter. In some embodiments, the implant is about 2.4 mm in diameter. In some embodiments, the implant is about 1 to about 2 mm in diameter. In some embodiments, the implant is about 1.5 mm in diameter. In some embodiments, the implant comprises dimensions of about 2.4 mm in total diameter and about 2.6 cm in total length. In some embodiments, the implant is about 1.5 mm in diameter. In some embodiments, the implant comprises dimensions of about 1.5 mm in total diameter and about 2.6 cm in total length.

In some embodiments, the core, the first layer, and any additional layer or layers comprising a biocompatible polymeric matrix, or a biocompatible polymeric matrix and a drug, are each independently about 0.5 to about 5 mm in diameter or thickness. In one embodiment, the core and layer or layers are each independently about 0.5 to about 3.5 mm. In some embodiments, the core and layer or layers are each independently about 0.5 to about 2 mm. In some embodiments, the core and layer or layers are each independently about 1 to about 2 mm. The thickness or diameter of the core may vary from the thickness of the layer or layers. If multiple layers are present, each layer may have the same thickness as the other layers, or each layer may have a different thickness from the other layers, or some layers may have the same thickness as other layers while some layers may have a different thickness from other layers. By “thickness” of a layer is meant the distance, as measured from the center of the implant, between the start of the layer and the end of the layer; for example, for a cylindrical implant with regular, annular layers, a layer that starts at 2 mm from the center and that ends at 3.5 mm from the center has a thickness of 1.5 mm.

Although the implant may be illustrated as having a core and one or more layers which are cylindrical or annular in cross-section, it is understood that the cross-section of the core and one or more layers may be oval, polygonal, star-shaped, irregular, or of uneven thickness.

In some embodiments, the various layers comprising a polymer and drug may comprise different polymers, or mixtures thereof, and different drugs or mixtures thereof.

### Drug Release

The release of drug from the implant is dependent on the rate of dissolution and on passive diffusion through the polymer matrix. Therefore, the surface area of the implant determines the rate of release. The release mechanism of the drug from the polymeric material also depends on the nature of the polymer and the drug. The drug diffuses through the polymer to the surrounding tissues and bodily fluids. Release can also occur through degradation or erosion of the polymer, in the case of an erodible or bioerodible polymer. The degradation or erosion of the polymer may occur through hydrolysis, by enzymatic degradation, or via other processes.

Drug release rates are also affected by washing of the implant prior to insertion into the individual. Washed implants maintain a more-stable release rate after insertion; unwashed implants may show a significantly higher burst release immediately after implant. A burst release may be detrimental to the individual, as local or systemic drug concentration rises from zero to a potentially supra-therapeutic level rapidly. Initial burst may also unnecessarily deplete the drug depot and shorten the duration of the release period. The implants may be washed with any solvent in which the drug dissolves, such as water, ethanol, isopropanol, etc. Washing may be followed by drying to remove the solvent. Drying may be followed by packaging and sterilization.

In some circumstances, an initial high dose of a drug is desirable, and in those circumstances washing of the implant can be omitted in order to provide for an initial burst as a loading dose. In certain embodiments of the implant, a layer of substantially pure pharmaceutical substance is placed on the outside of the implant, for an enhanced loading dose (initial dose).

In a non-limiting example, the extruded implant can be cut into implants of appropriate length, such as 2.6 cm. The extrudate may be, optionally, washed, e.g., with 95% ethanol at room temperature for 30 min to remove surface drug. The washed implants can be dried (e.g., air dried at room temperature for 30 min, then forced air at 40° C. for 1 hour, followed by vacuum drying at 30° C. for 24 hours) to remove residual ethanol. Implants may be placed in moisture barrier foil pouches, heat-sealed and then sterilized using gamma irradiation (2.9-3.1 Mrads).

In some embodiments, the implants provide an approximately constant level of drug over a period of time in the plasma of the subject or individual. In one embodiment, an approximately constant level of drug varies by no more than about ±30% over a day, over a week, over a month, over three months, over six months, or over nine months, as compared to the mean or average plasma level over that time period. In some embodiments, an approximately constant level of drug varies by no more than about ±20% over a day, over a week, over a month, over three months, over six months, or over nine months, as compared to the mean or average plasma level over that time period. In some embodiments, an approximately constant level of drug varies by no more than about ±10% over a day, over a week, over a month, over three months, over six months, or over nine months, as compared to the mean or average plasma level over that time period. An “approximately constant release rate” indicates that an approximately constant level of the pharmaceutical substance is released from the implant over a period of time, such as over a day, over a week, over a month, over three months, over six months, or over nine months. In some embodiments, the approximately constant release rate varies by no more than about ±30%, ±20%, or ±10% over the time period indicated, of the average or mean release. An approximately constant release rate is preferred in order to achieve a steady state plasma level. The average or mean release can be calculated starting after the first week after implantation, in order to exclude the initial burst period.

By “essentially constant” is meant that for about 80% of the extended period of time, the concentration of drug in blood plasma is within about three, about two, or preferably about one standard deviation of the mean blood plasma level. Measurements of the blood plasma level can be performed hourly, twice a day, daily, twice a week, weekly, every two weeks, monthly, or at any other periodic interval for determination of the mean plasma levels. For example, if the mean blood plasma level of a drug sampled at weekly intervals is 2.0 ng/ml, and one standard deviation of the measurement is ±0.1 ng/ml, then blood plasma levels that fall within about ±0.3 ng/ml, about ±0.2 ng/ml, or preferably about ±0.1 ng/ml for about 80% of the measurements are considered essentially constant. By “extended periods of time” is meant from about 3 months to about 1 year, or longer, e.g., at least about 3, about 6, about 9, about 12, about 15, about 18, about 21, or about 24 months or more. The mean blood plasma level can be calculated starting after the first week after implantation, in order to exclude the initial burst period.

In embodiments where an initial burst or an initial loading dose is desired (such as embodiments where excess pharmaceutical substance is not washed off of the surface of the implant, or embodiments where the implant is surrounded by a layer of pure drug), the period during which the initial burst or initial loading dose occurs is excluded from the calculation of steady-state plasma levels or steady-state release rates, approximately constant plasma levels or approximately constant release rates, or essentially constant plasma levels or essentially constant release rates. The initial burst period or initial loading dose period ends when the release rate or plasma level falls within the ranges as specified above for steady-state, approximately constant, or essentially constant.

The implants as disclosed herein show particularly good rates of release of Compound A, a small hydrophilic peptide. Delivery of hydrophilic substances from implants can be difficult, as rapid release of the contents may occur when an implant containing the hydrophilic substance comes into contact with aqueous interstitial fluid in the body. Further, administration of therapeutic peptides for long periods of time is challenging, due to rapid breakdown of small peptides in circulation by peptidases and proteases. The half-life of a peptide drug in circulation tends to be relatively short, often on the order of hours. As shown in Example 1, the disclosed implant provides controlled release resulting in a sustained and significant plasma level of Compound A in rats over 2000 hours (about 83 days, or nearly three months) after implantation. Achieving controlled release of a hydrophilic peptide drug in blood for nearly three months, as in the current disclosure, is a significant result. In view of these results, the disclosed implants can provide important benefits to individuals suffering from pruritis in need of long-term treatment.

### Kinetics of Drug Delivery

Drug delivery can have a controlled release during the life of implant. If a laminate implant, which comprises a core and one or more layers, is used, varying the concentration of kappa-opioid receptor agonist in the core and/or different layers can be used to modulate the rate of drug delivery over time. In one embodiment, the implant displays a generally linear release of kappa-opioid receptor agonist over time. In some embodiments, kappa-opioid receptor agonist release from the implant is approximately constant or essentially constant over the lifetime of the implant, or for a specified period within the lifetime of the implant. In an implant with a core and at least one layer which contain kappa-opioid receptor agonist, the agonist is released from the implant, layer by layer, from outer-most to inner-most layers. However, each layer will have a diameter and surface area smaller than the layer outside it. Thus, layers closer to the interior will need to have a higher concentration of kappa-opioid receptor agonist than more outer layers, in order to maintain an approximately constant or essentially constant rate of agonist release. In some embodiments, the concentrations of kappa-opioid receptor agonist layer-by-layer can be designed to create different rates of agonist release. For example, if each layer contains the same or a lower kappa-opioid receptor agonist concentration than the adjoining outer-more layer, this will result in a tapered, ever-decreasing rate of agonist delivery. Modulating concentrations of kappa-opioid receptor agonist layer-by-layer can also produce a slow rise in agonist delivery over the lifetime of the implant or a specified period during the lifetime of the implant. Alternating layers of relatively high and low concentrations of kappa-opioid receptor agonist can produce a pulsed rate of agonist delivery that rises and falls over time.

In one embodiment, more than one implant may be inserted into an individual to achieve a desired level of drug concentration in the blood plasma. The level of drug delivery is preferably within the therapeutic range of the drug and lower than a level that might cause toxicity. In one embodiment, the implant can comprise multiple drugs. In one embodiment, the multiple drugs are integrated into the implant and released layer by layer to maintain steady-state levels of each drug throughout the duration of implant. In some embodiments, the drugs are distributed in varying concentrations layer by layer so that drug delivery may occur in waves, with a higher dosage of one drug released, followed by a higher dosage of another drug over time.

The implant may be designed to provide a steady-state concentration of drug in the blood plasma. The implant may be designed such that the resulting concentration of drug in the blood plasma remains essentially constant over extended periods of time. The implant may be designed such that the resulting concentration of drug in the blood plasma remains approximately constant over extended periods of time.

In another aspect, the disclosure provides an implant for treating pruritus, comprising kappa-opioid receptor agonist and a biocompatible polymeric matrix, wherein when said implant is implanted subcutaneously in a mammal, said kappa-opioid receptor agonist is continuously released in vivo over a sustained period of time through pores that open to the surface of said matrix at a steady state rate.

In some embodiments, the implants are configured to release about 0.01 mg of kappa-opioid receptor agonist per day. In some embodiments, the implants are configured to release at least about 0.01 mg per day, such as about 0.01 to about 5 mg per day, about 0.01 to about 4 mg per day, about 0.01 to about 3 mg per day, about 0.01 to about 2 mg per day, about 0.01 to about 1 mg per day, about 0.01 to about 0.5 mg per day, about 0.01 to about 0.25 mg per day, about 0.01 to about 0.1 mg per day, about 0.01 to about 0.05 mg per day, or about 0.01 to about 0.02 mg of kappa-opioid receptor agonist per day.

In some embodiments, the implants are configured to release about 0.05 mg of kappa-opioid receptor agonist per day. In some embodiments, the implants are configured to release at least about 0.05 mg per day, such as about 0.05 to about 5 mg per day, about 0.05 to about 4 mg per day, about 0.05 to about 3 mg per day, about 0.05 to about 2 mg per day, about 0.05 to about 1 mg per day, about 0.05 to about 0.5 mg per day, about 0.05 to about 0.25 mg per day, or about 0.05 to about 0.1 mg of kappa-opioid receptor agonist per day.

In some embodiments, the implants are configured to release about 0.1 mg of kappa-opioid receptor agonist per day. In some embodiments, the implants are configured to release at least about 0.1 mg per day, such as about 0.1 to about 5 mg per day, about 0.1 to about 4 mg per day, about 0.1 to about 3 mg per day, about 0.1 to about 2 mg per day, about 0.1 to about 1 mg per day, about 0.1 to about 0.5 mg per day, about 0.1 to about 0.25 mg per day, or about 0.1 to about 0.2 mg of kappa-opioid receptor agonist per day.

In some embodiments, the approximate rate of kappa-opioid receptor agonist release is about 0.01 mg per day, 0.02 mg per day, 0.03 mg per day, 0.04 mg per day, 0.05 mg per day, 0.75 mg per day, 0.1 mg per day, 0.2 mg per day, 0.3 mg per day, about 0.4 mg per day, about 0.5 mg per day, about 0.6 mg per day, about 0.7 mg per day, about 0.8 mg per day, about 0.9 mg per day, or about 1.0 mg per day.

When implanted subcutaneously, implants disclosed herein continuously release kappa-opioid receptor agonist for an extended period of time with a pseudo or near zero order release rate. In some embodiments, the initial burst of kappa-opioid receptor agonist released in vivo after implantation is reduced or minimized by prewashing the implants before implantation to remove surface kappa-opioid receptor agonist. Prewashing may be performed in any solution in which kappa-opioid receptor agonist is soluble, for example 30 minutes in ethanol. The release rate can be altered by modifying the percent kappa-opioid receptor agonist loading, porosity of the matrix, structure of the implant, or hydrophobicity of the matrix, or by adding a hydrophobic coating to the exterior of the implant. The implants can deliver kappa-opioid receptor agonist without the need for external medical equipment such as intravenous lines or pumps.

### Exemplary Polymers

In some embodiments, the implants can comprise a single biocompatible polymeric matrix material blended with kappa-opioid receptor agonist. In some embodiments, the implants can be heterogeneous, that is, the implants can comprise multiple layers having different compositions. For example, the implants can comprise a core comprising a core biocompatible polymeric matrix, surrounded by one or more layers comprising a layer biocompatible polymeric matrix. At least one of the core and the one or more layers additionally comprises a kappa-opioid receptor agonist. Optionally, at least one of the core and the one or more layers can comprise one or more pharmaceutical substances. The one or more pharmaceutical substances can be in the core or the same layer as the kappa-opioid receptor agonist, or can be in a different location than the kappa-opioid receptor agonist. That is, whether the kappa-opioid receptor agonist is in the core or in the one or more surrounding layers, the additional pharmaceutical substance or substances can be in any of the core or the one or more surrounding layers.

The core and layer biocompatible polymeric matrices may be the same or different. The core or any layer may also comprise a mixture of two or more biocompatible polymers; the core and the various layers may contain different mixtures of polymers.

As used herein, a “polymer” or “polymeric material” means a macromolecule comprising repeating monomer units or co-monomer units. The polymer may be bioerodible or non-bioerodible. The polymer may be a homopolymer, copolymer, terpolymer, or may contain more than three monomers.

Exemplary polymers that can be used for making the implant include: acrylics, agarose, alginate, and combinations, cellulose ethers, collagen, copolymers containing poly(ethylene glycol) and polybutylene terephthalate segments (PEG/PBT) (PolyActive™), copolymers of poly(lactic) and glycolic acid, copolymers thereof with poly(ethylene glycol), derivatives and mixtures thereof, dextran, dextrose, elastin, epoxides, ethylene vinyl acetate (EVA copolymer), fluoropolymers, gelatin, hydroxypropylmethylcellulose, maleic anhydride copolymers, methyl cellulose and ethyl cellulose, non-water soluble cellulose acetate, non-water soluble chitosan, non-water soluble hydroxyethyl cellulose, non-water soluble hydroxypropyl cellulose, peptides, PLLA-polyglycolic acid (PGA) copolymer (also known as poly-L-lactic acid-co-glycolic acid, or PLGA), poly (L-lactic acid), poly(2-ethoxyethyl methacrylate), poly(2-hydroxyethyl methacrylate), poly(2-methoxyethyl acrylate), poly(2-methoxyethyl methacrylate), poly(acrylamide), poly(alginic acid), poly(amino acids), poly(anhydrides), poly(aspartic acid), poly(benzyl glutamate), poly(beta-hydroxybutyrate), poly(caprolactone), poly(D,L-lactic acid), poly(D,L-lactide)(PLA), poly(D,L-lactide-co-caprolactone)(PLA/PCL) and poly(glycolide-co-caprolactone) (PGA/PCL), poly(D,L-lactide-co-glycolide) (PLA/PGA), poly(etherurethane urea), poly(ethyl glutamate-co-glutamic acid), poly(ethylene carbonate), poly(ethylene glycol), poly(ethylene-co-vinyl alcohol), poly(glutamic acid), poly(glutamic acid-co-ethyl glutamate), poly(glycolic acid), poly(glycolide-co-trimethylene carbonate) (PGA/PTMC), poly(hydroxypropyl methacrylamide), poly(imino carbonates), poly(leucine), poly(leucine-co-hydroxyethyl glutamine), poly(L-lactide-co-D,L-lactide) (PLLA/PLA), poly(L-lactide-co-glycolide)(PLLA/PGA), poly(lysine), poly(ortho esters), poly(orthoesters), poly(oxaamides), poly(oxaesters), poly(phosphate ester), poly(phosphazene), poly(phospho esters), poly(phosphoesters), poly(propylene carbonate), poly(propylene glycol), poly(pyrrole), poly(tert-butyloxy-carbonylmethyl glutamate), poly(tetramethylene glycol), poly(trimethylene carbonate), poly(ureas), poly(urethanes), poly(urethane-ureas), poly(vinyl alcohol), poly(vinyl alcohol-co-vinyl acetate), poly(vinylpyrrolidone) (PVP), poly[(97.5% dimethyl-trimethylene carbonate)-co-(2.5% trimethylene carbonate)], polyacrylic acid, polyalkylene oxides, polyamides, polycaprolactone (PCL) poly-(hydroxybutyrate-co-hydroxyvalerate) copolymer (PHBV), polycaprolactone (PCL), polycaprolactone co-butylacrylate, polydepsipeptides, polydioxanone (PDS), polyesters, polyethylene glycol, polyethylene oxide (PEO), polyethylene terephthalate (PET), polyglycolic acid and copolymers and mixtures thereof such as poly(L-lactide) (PLLA), polyglycolic acid[polyglycolide (PGA)], polyhydroxybutyrate (PHBT) and copolymers of polyhydroxybutyrate, polyiminocarbonates, polylactic acid, polymethacrylic acid, polyolefins, polyphosphazene polymers, polypropylene fumarate, polysaccharides such as hyaluronic acid, polytetrafluoroethylene (PTFE Teflon®), polyurethanes, silicones, tyrosine-derived polyarylates, tyrosine-derived polycarbonates, tyrosine-derived polyiminocarbonates, tyrosine-derived polyphosphonates, urethanes, and combinations, derivatives and mixtures thereof.

Exemplary erodible or bioerodible polymers that can be used for making the implant include erodible or bioerodible forms of polyamide, aliphatic polycarbonates, polyalkylcyanoacrylate, polyalkylene oxalates, polyanhydride, polycarboxylic acid, polyester, poly(hydroxybutyrate), polyimide, poly(iminocarbonate), polycaprolactone (PCL), poly-D,L-lactic acid (DL-PLA), polydioxanone, poly(glycolic acid), poly-L-lactic acid (L-PLA), poly-L-lactic acid-co-glycolic acid (PLGA), polyorthoester, polyphosphazenes, and polyphosphoester, poly(trimethylene carbonate), and derivatives and mixtures thereof. The polymer may also be formed from a material selected from the group consisting of cellulose ester, polybutylene terephthalate, polycarbonate, polyester, polyether ether ketone, polyethylene-co-tetrafluoroethylene, polymethylmethacrylate, polyolefin, polypropylene, polysulfones, polytetrafluoroethylene, polyurethane, polyvinylchloride, polyvinylidene fluoride, silicone, and derivatives and combinations thereof.

Additional representative examples of the polymer for use in the implants include, but are not limited to, ABS resins, acrylic polymers and copolymers, acrylonitrile-styrene copolymers, alkyd resins, and carboxymethyl cellulose, and ethylene-vinyl acetate copolymers, cellophane, cellulose butyrate, cellulose acetate butyrate, cellulose acetate, cellulose ethers, cellulose nitrate, cellulose propionate, copolymers of vinyl monomers with each other and olefins, ethylene-methyl methacrylate copolymers, epoxy resins, ethylene vinyl alcohol copolymer (commonly known by the generic name EVOH or by the trade name EVAL), poly(glyceryl sebacate), poly(glycolic acid-co-trimethylene carbonate), poly(hydroxybutyrate-co-valerate), poly(hydroxyvalerate), poly(lactide-co-glycolide), poly(propylene fumarate), poly(trimethylene carbonate), polyacrylonitrile, polyamides, such as Nylon 66 and polycaprolactam, polycarbonates, polycyanoacrylates, polydioxanone, polyesters, polyethers, polyimides, polyisobutylene and ethylene-alphaolefin copolymers, polyoxymethylenes, polyphosphoester urethane, polyvinyl ketones, polyvinyl aromatics, such as polystyrene, polyvinyl esters, such as polyvinyl acetate, polyvinyl ethers, such as polyvinyl methyl ether, polyvinylidene halides, such as vinylidene fluoride based homo- or copolymer under the trade name Solef™ or Kynar™, for example, polyvinylidene fluoride (PVDF) or poly(vinylidene-co-hexafluoropropylene) (PVDF-co-HFP) and polyvinylidene chloride, rayon, rayon-triacetate, silicones, vinyl halide polymers and copolymers, such as polyvinyl chloride, copolymers of these polymers with poly(ethylene glycol) (PEG), or combinations thereof.

In some embodiments, the polymer can be copolymers of poly(lactic) and glycolic acid, poly(anhydrides), poly(D,L-lactic acid), poly(D,L-lactide), poly(D,L-lactide-co-glycolide), poly(ethylene carbonate), poly(glycolic acid), poly(glycolide), poly(L-lactic acid), poly(L-lactide), poly(L-lactide-co-glycolide), poly(ortho esters), poly(oxaamides), poly(oxaesters), poly(phosphazenes), poly(phospho esters), poly(phosphoesters), poly(propylene carbonate), poly(trimethylene carbonate), poly(tyrosine derived carbonates), poly(tyrosine derived iminocarbonates), poly(tyrosine derived arylates), copolymers of these polymers with poly(ethylene glycol) (PEG), or combinations thereof.

Examples of non-bioerodible polymers useful in the present implants include poly(ethylene-co-vinyl acetate) (EVA), polyvinylalcohol and polyurethanes, such as polycarbonate-based polyurethanes.

A preferred polymer for the implants is ethyl vinyl acetate (EVA).

In some embodiments wherein the implant comprises EVA, or where the implant comprises a core and one or more additional layers comprising EVA, the vinyl acetate content can be about 2 to about 40, about 10 to about 35, about 30 to about 35%, or about 33% by weight.

The EVA-kappa-opioid receptor agonist blend of the first layer of the implants generally comprise about 10% to about 85%, such as about or at least about 10, 20, 30, 40, 50, 55, 60, 65, 70, 75, 80, or 85% kappa-opioid receptor agonist, such as about 25% to about 50% kappa-opioid receptor agonist, or about 50% to about 75% kappa-opioid receptor agonist. In one embodiment, the EVA-kappa-opioid receptor agonist blend of the first layer of the implant comprises about 50% kappa-opioid receptor agonist and about 50% EVA. In some embodiments, the EVA-kappa-opioid receptor agonist blend of the first layer of the implant comprises about 66.7% kappa-opioid receptor agonist and about 33.3% EVA. In some embodiments, the EVA-kappa-opioid receptor agonist blend of the first layer of the implant comprises about 75% kappa-opioid receptor agonist and about 25% EVA. In various embodiments, the sustained period of time for kappa-opioid receptor agonist release is from about 3 months to about 1 year, or longer, e.g., at least about 3, 6, 9, or 12 months.

Either the core or any layer of the implant can comprise a single type of polymer or a mixture of two or more polymers. A mixture of two polymers may modulate the release rate of the drug. It is desirable that an effective therapeutic amount of the drug be released from the implant for a reasonably long period of time. U.S. Pat. No. 6,258,121 to Yang et al. disclosed a method of altering the release rate by blending two polymers with differing release rates and incorporating them into a single layer; this technique can also reduce burst release of drug upon implant.

Where appropriate, a coating that is impermeable to the kappa-opioid receptor agonist is placed over at least a portion of the implant to further regulate the rate of release. Where appropriate, the implant does not have any coating that is impermeable to the kappa-opioid receptor agonist.

### Exemplary Additional Pharmaceutical Substances

As used herein, a “drug” or “pharmaceutical substance” is any biologically active agent or other substance that has therapeutic value to a living organism. Additional pharmaceutical substances can be included in the implants comprising a kappa-opioid receptor agonist. These additional pharmaceutical substances include additional kappa-opioid receptor agonists, mu-opioid receptor agonists, mu-opioid receptor antagonists, mixed mu-opioid receptor agonists/antagonists such as buprenorphine, delta-opioid receptor agonists, delta-opioid receptor antagonists, anti-inflammatory drugs, and steroids.

In some embodiments, the additional pharmaceutical substance or substances are independently selected from the group consisting of buprenorphine and fentanyl.

### Insertion and Removal of Implants

In one method of this disclosure, the implant is administered by subcutaneous implantation, and preferably placed in the interstitial space between the skin and the muscle layer. In various embodiments, the implants are subcutaneously implanted at a site selected from a group consisting of the upper arm, scapular region, the back, the leg and the abdomen. Before implantation, the individual may be lightly anesthetized, e.g., with isoflurane or other anesthetic known in the art, and/or may have topical, transdermal, or subcutaneous anesthetic applied at the site of implantation. A small incision can be made through the skin and a trocar inserted subcutaneously, then loaded with one implant. The stylet can be inserted to hold the implant in place and the trocar carefully removed, leaving the implant in the subcutaneous space. Each site can be sutured closed and examined later. Complications such as skin irritation, inflammation, infection or other site-specific adverse effects can be monitored and treated, e.g., with antibiotics, as needed.

In various embodiments, the implant can be left in the body for up to about three months; for example, it can be left in the body for about one month to about two months, about two months to about three months, or for about one month to about three months. In various embodiments, the implant can be left in the body for up to about six months; for example, it can be left in the body for about one month to about six months, about three months to about six months, or for about five months to about six months. In various embodiments, the implant can be left in the body for up to about one year; for example, it can be left in the body for about six months to about one year or about nine months to about one year. The period of sustained release of drug into the body can be from about 3 months to about 1 year, or longer, e.g., at least about 3 months to about 6 months, at least about 6 months to about 9 months, at least about 9 months to about a year, at least about 12 months to about 18 months, at least about 15 months to about 24 months, at least about 18 months to about 24 months, at least about 21 months to about 24 months, or up to about 24 months. In some embodiments the implant can be left in the body for up to 1 year. Implants may be removed from the body at the end of the treatment period, through an incision, e.g., a 3-mm incision, using forceps.

A second implant may, for example, be used to deliver a pharmaceutical substance to counteract any adverse effects caused by a drug released from a first implant.

Multiple implants may be inserted into a single individual to regulate the delivery of a single drug, or to deliver several drugs.

### Indications for Usage

One or more implants as disclosed herein can be used to treat pruritus. One or more implants as disclosed herein can be used to treat chronic pruritus, that is, pruritus lasting more than six weeks. One or more implants as disclosed herein can be used to treat uremic pruritus. One or more implants as disclosed herein can be used to treat pruritus arising from causes such as pruritus associated with chronic liver disease, notalgia paresthetica, eczema, dermatitis, atopic dermatitis, psoriasis, burns, or scarring.

In additional embodiments, the disclosure embraces implants comprising a kappa opioid receptor agonist for use in treating pruritus. In additional embodiments, the disclosure embraces implants comprising a kappa opioid receptor agonist for use in treating uremic pruritus. In additional embodiments, the disclosure embraces implants comprising a kappa opioid receptor agonist for use in treating pruritus arising from causes such as pruritus associated with chronic liver disease, notalgia paresthetica, eczema, dermatitis, atopic dermatitis, psoriasis, burns, or scarring.

In additional embodiments, the disclosure embraces use of a kappa opioid receptor agonist and a polymer as disclosed herein for the manufacture of an implant for treatment of pruritus. In additional embodiments, the disclosure embraces use of a kappa opioid receptor agonist and a polymer as disclosed herein for the manufacture of an implant for treatment of uremic pruritus. In additional embodiments, the disclosure embraces use of a kappa opioid receptor agonist and a polymer as disclosed herein for the manufacture of an implant for treatment of pruritus arising from causes such as pruritus associated with chronic liver disease, notalgia paresthetica, eczema, dermatitis, atopic dermatitis, psoriasis, burns, or scarring.

### Example 1

Blood Plasma Levels Resulting from Implants of Compound a in Rats

Ten coextruded implants were prepared having a core comprising 50% of Compound A (as a salt) and 50% ethylene vinyl acetate, and a shell comprising 3% Compound A and 97% ethylene vinyl acetate. The implants measured 26 mm length×1.5 mm diameter, weighed 48 mg, and contained 17.5 mg Compound A. After extrusion, the implants were washed, dried, packaged and sterilized by E-beam irradiation at 25 KGy.

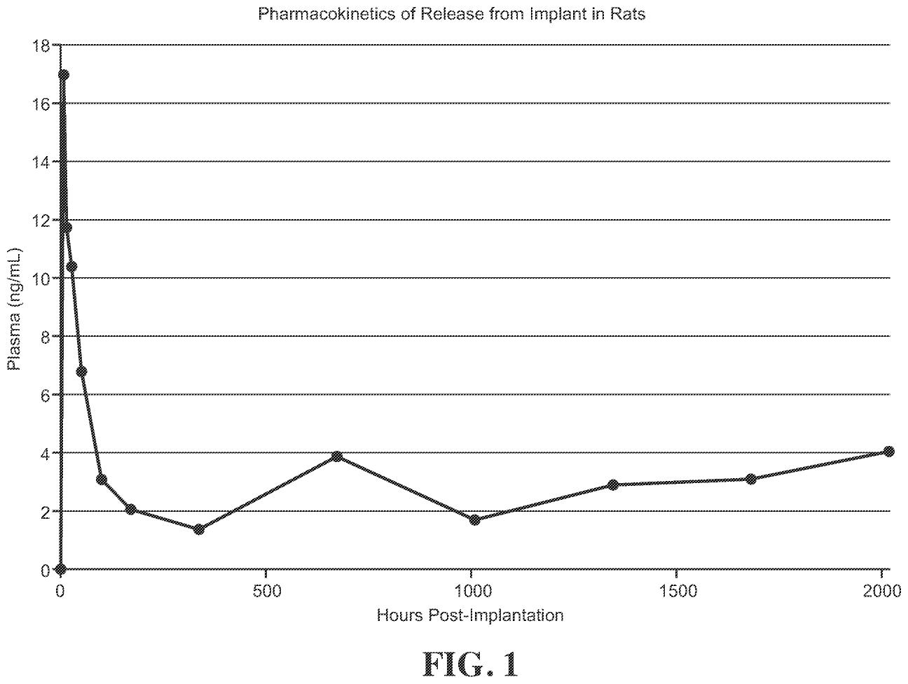

The implants were implanted into rats (one implant per rat), and blood plasma levels were measured. The results are shown in FIG. 1. After an initial peak from drug on the implant surface, the rat plasma levels were sustained within a range of about 1.4 to about 4 ng/ml for about 2,000 hours (about 83 days).

### Example 2

Extended Efficacy of Compound a in the Mouse Itch Model by Chronic Delivery with Mouse Prototype EVA Implants

Compound A implants were formulated in ethylene-vinyl acetate (EVA) matrices for testing in male C₅₇BL/6 mice. Because of the small size of the mouse, test implant prototypes (25 mm×1.5 mm) containing a limited quantity of the drug (10 mg) were used for this mouse model study. The mouse implants had a core comprising 50% of Compound A (as a salt) and 50% ethylene vinyl acetate, and a shell comprising 3% Compound A and 97% ethylene vinyl acetate. After extrusion, the implant prototypes were washed, dried, packaged and sterilized by E-beam irradiation at 25 KGy.

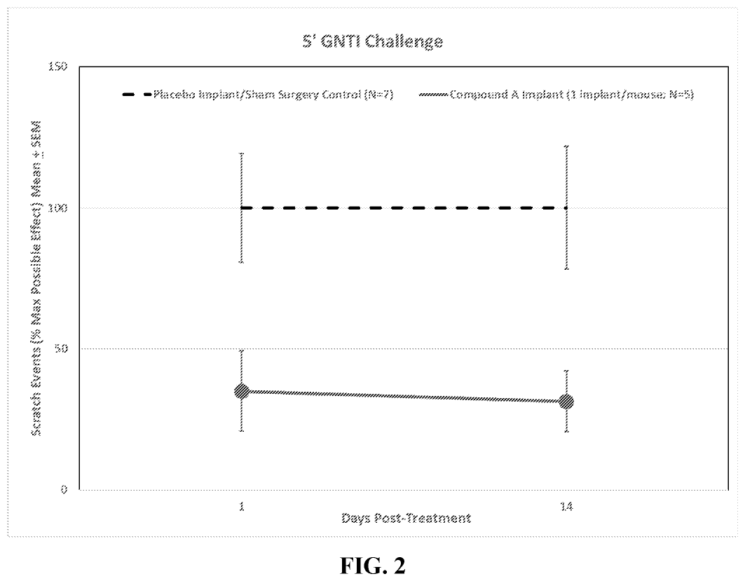

Following the implantation of one Compound A implant in each mouse, the treated mice with drug-containing implants (N=5) and untreated control mice with placebo implants or sham surgery (N=7) were challenged with 5′-guanidinonaltrindole (5′GNTI) at Days 1, 14 and 28 post-implantation. Scratch events were monitored. The results, shown in FIG. 2, indicated that the Compound A prototype implants at the dose of 1 implant per mouse provided sustained itch suppression for up to 14 days post-implantation, with scratching behavior returning to control levels by day 28 for the animals.

### Example 3

Pharmacokinetic Assessment of Compound a Mouse Plasma Concentration from Subcutaneous Acute Injection and from EVA Mouse Prototype Implants

Male C57BL/6 mice were sampled for pharmacokinetic analysis following subcutaneous acute injection with Compound A in saline (0.3 mg/kg drug was administered, using an injection volume of 10 mL/kg), or following implantation with one Compound A implant in each mouse. The implants used were as described in Example 2.

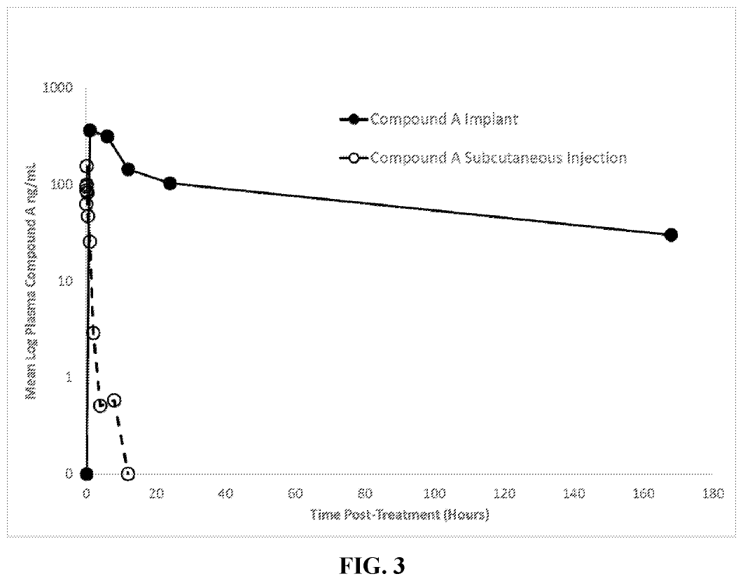

The results, shown in FIG. 3, indicate that over the first 14 days post-implantation, the Compound A mouse prototype implants provided plasma concentrations of Compound A that were well above the levels estimated for therapeutic anti-pruritus activity with an acute 0.3 mg/kg subcutaneous injection.

### Example 4

### Simulated Plasma Levels

Simulated plasma levels were calculated for implants containing Compound A, and for an intravenous bolus dose of CR-845, a kappa opioid receptor agonist. CR-845 is generically known as difelikefalin, and sold under the brand name Korsuva®.

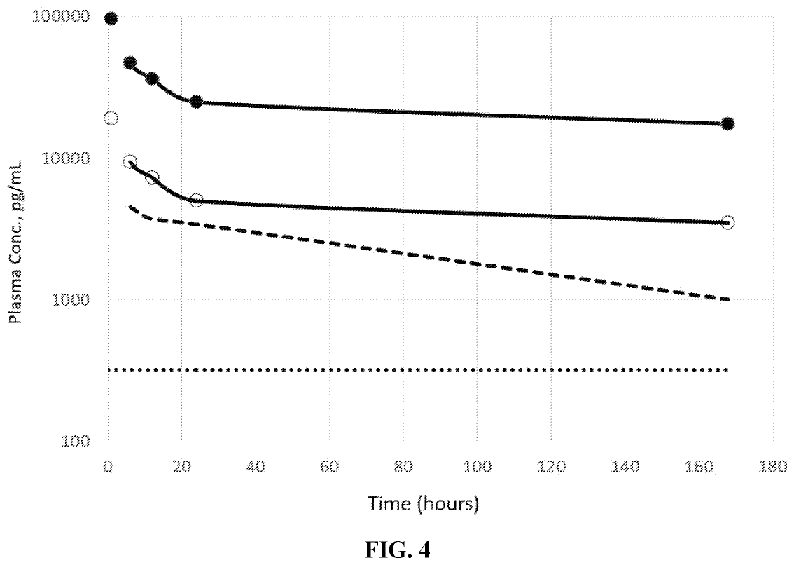

FIG. 4 shows simulated human plasma levels over 1 week after an implant with Compound A (0.5 mg/day (filled circles) or 0.1 mg/day (open circles)), and for CR-845, as a 0.08 mg IV bolus (dashed line), as compared to the plasma level of difelikefalin (320 pg/mL) required for 75% kappa opioid receptor occupancy (dotted line). Mouse subcutaneous data was used in the simulation to define the pharmacokinetic parameters for distribution and elimination by allometrically scaling to humans (factor 13.7).

### Example 5

### Compound A-Containing Implants Reduce Scratching Events in Mice Over an 84-Day Period

Male C57BL/6 mice (8-14 wks old, N=7-8) were each implanted with one Compound A-containing implant (26×2.6 mm, 50% loading, 60 mg Compound A/implant). Controls were untreated mice or mice implanted with placebo (EVA-only) implants (N=5-7). On Days 28, 56 and 84 post-implantation, the mice were transferred to behavioral rooms during the light cycle, at least 2 hours prior to challenge with the pruritogen 5′-GNTI to minimize stress effects on grooming and movement. 5′GNTI (0.1 mg/kg) was subcutaneously injected just caudal to the head/neck, and the mice were immediately placed in an observation chamber (25×25 cm) with plexiglas walls and light-colored melamine flooring and video recorded from above for up to 90 minutes after pruritogen challenge, and then removed and placed back into their home cages in the housing colony. The video recordings were subsequently analyzed for the quantity of scratching behavior, classified as either a forepaw scratching bout behind the ears, or hind paw scratching bout of the flank/back of the animal, over the initial 30 minutes of observation.

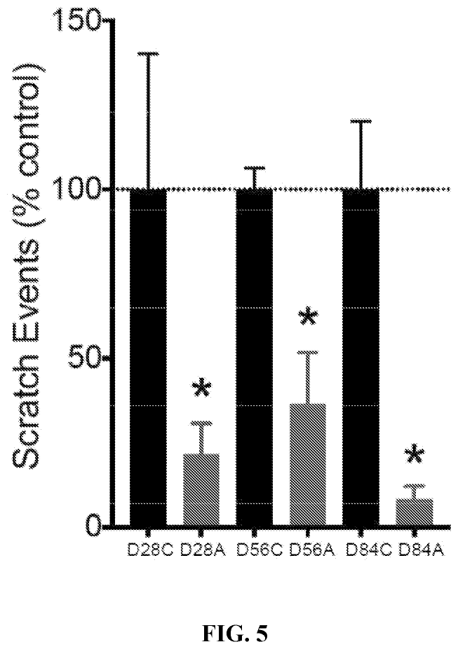

FIG. 5 shows reduced scratch events in mice with Compound A-containing implants, versus control mice. (D28C=control mice at day 28, D28A=mice with Compound A implants at day 28, D56C=control mice at day 56, D56A=mice with Compound A implants at day 56, D84C=control mice at day 84, D84A=mice with Compound A implants at day 84).

Although the foregoing disclosed compositions, articles of manufacture, implants, methods, kits, and other disclosures have been described in some detail by way of illustration and examples for purposes of clarity of understanding, it will be apparent to those skilled in the art that certain changes and modifications may be practiced without departing from the spirit and scope of the disclosure. Therefore, the description should not be construed as limiting the scope of the invention.

All publications, patents, and patent applications cited herein are hereby incorporated by reference in their entirety.

What is claimed is:

1. A method of treating pruritus in an individual, comprising:
administering an implant comprising a sustained release composition to the individual, wherein the sustained release composition comprises a kappa-opioid receptor agonist and a biocompatible polymeric matrix,
wherein the sustained release composition is configured to release a therapeutically effective amount of the kappa-opioid receptor agonist after administration.

2. The method of claim 1, wherein the kappa-opioid receptor agonist comprises a peptide.

3. The method of claim 2, wherein the kappa-opioid receptor agonist comprises a compound of formula I: 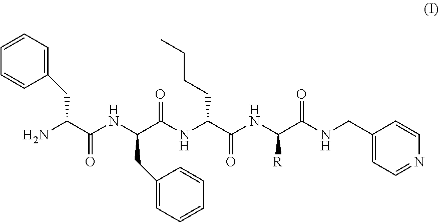
or a pharmaceutically acceptable salt or stereoisomer thereof,
wherein R is 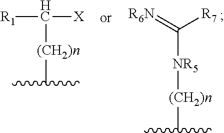
wherein n is an integer from 1 to 4;
X is —NR₂R₃ or —N^{⊕} R₂R₃R₄;
each of R₁, R₂, R₃, and R₄ is independently hydrogen, C₁-C₅ alkyl, C₁-C₅ substituted alkyl, C₂-C₅ alkenyl, C₂-C₅ substituted alkenyl, C₂-C₅ alkynyl, C₂-C₅ substituted alkynyl, C₃-C₆ cycloalkyl, C₆-C₁₀ aryl, C₆-C₁₀ substituted aryl, or —C₁-C₅ alkyl-C₆-C₁₀ aryl;
R₇ is hydrogen, C₁-C₅ alkyl, C₁-C₅ substituted alkyl, C₂-C₅ alkenyl, C₂-C₅ substituted alkenyl, C₂-C₅ alkynyl, C₂-C₅ substituted alkynyl, C₃-C₆ cycloalkyl, C₆-C₁₀ aryl, C₆-C₁₀ substituted aryl, —C₁-C₅ alkyl-C₆-C₁₀ aryl, or —NR₈R₉;
each of R₅, R₆, R₈, and R₉ is independently hydrogen, C₁-C₅ alkyl, C₁-C₅ substituted alkyl, C₁-C₅ alkenyl, C₁-C₅ substituted alkenyl, C₁-C₅ alkynyl, C₁-C₅ substituted alkynyl, C₃-C₆ cycloalkyl, C₆-C₁₀ aryl, C₆-C₁₀ substituted aryl, or —C₁-C₅ alkyl-C₆-C₁₀ aryl; or
alternatively, R₅ and R₉ taken together with the nitrogen atoms to which they are attached form a heterocyclic ring; or
alternatively, R₆ and R₉ taken together with the nitrogen atoms to which they are attached form a heterocyclic ring.

4. The method of claim 3, wherein R is:

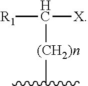

5. The method of claim 3, wherein R is:

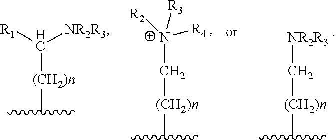

6. The method of claim 3, wherein R is:

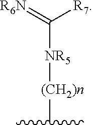

7. The method of claim 3, wherein R is:

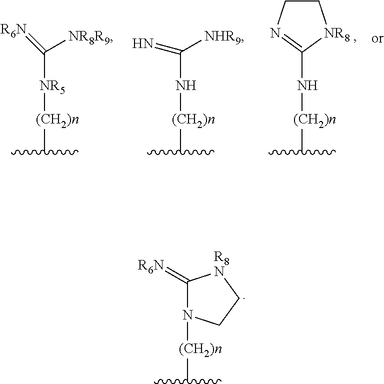

8. The method of claim 3, wherein R is:

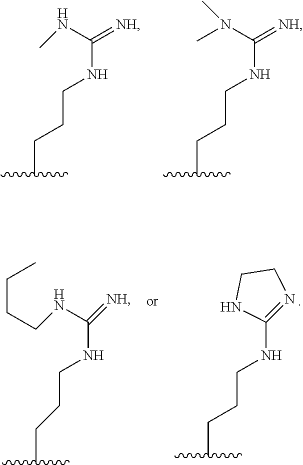

9. The method of claim 3, wherein R is

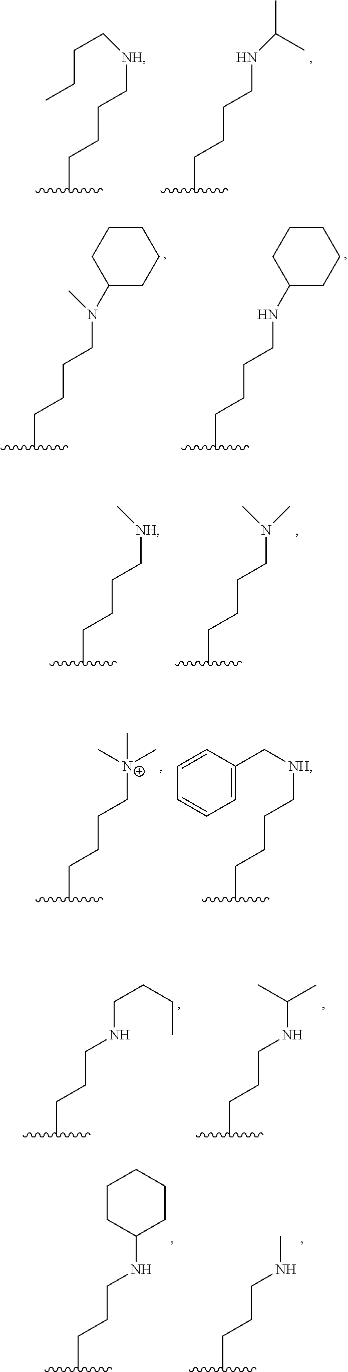

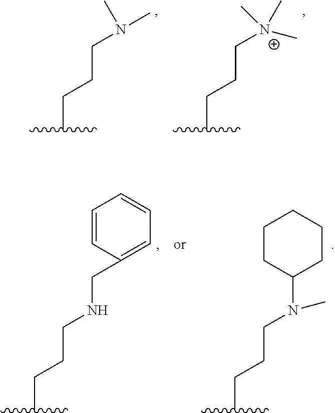

10. The method of claim 3, wherein R is:

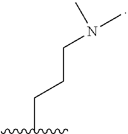 [SMILES: *CCCN(C)C]

11. The method of any one of claims 1-10, wherein the biocompatible polymeric matrix comprises ethylene vinyl acetate (EVA) copolymer, crosslinked poly(vinyl alcohol), poly(hydroxy ethylmethacrylate), an acyl substituted cellulose acetate, a hydrolyzed alkylene-vinyl acetate copolymer, polyvinyl chloride, polyvinyl acetate, a polyvinyl alkyl ether, polyvinyl fluoride, polycarbonate, polyurethane, polyamide, polysulphone, styrene acrylonitrile copolymer, crosslinked poly(ethylene oxide), a poly(alkylene), poly(vinyl imidazole), a poly(ester), poly(ethylene terephthalate), polyphosphazene, a chlorosulphonated polyolefin, poly-lactide (PLA), poly-glycolide (PGA), poly(lactic-co-glycolic acid) (PLGA), or combinations thereof.

12. The method of any one of claims 1-10, wherein the biocompatible polymeric matrix comprises ethylene vinyl acetate (EVA) copolymer.

13. The method of claim 12, wherein the EVA copolymer comprises about 20% to about 40% vinyl acetate of the total weight of the copolymer.

14. The method of claim 12, wherein the EVA copolymer comprises about 33% vinyl acetate of the total weight of the copolymer.

15. The method of any one of claims 1-14, wherein the kappa-opioid receptor agonist comprises about 10% to about 85% of the total weight of the sustained release composition.

16. The method of any one of claims 1-14, wherein the kappa-opioid receptor agonist comprises about 30% about 70% of the total weight of the sustained release composition.

17. The method of any one of claims 1-16, wherein the implant is a rod-shaped device having a diameter of about 0.5 mm to about 10 mm, and a length of about 0.5 cm to about 10 cm.

18. The method of any one of claims 1-16, wherein the implant is a rod-shaped device having a diameter of about 2 mm to about 3 mm, and a length of about 2 cm to about 4 cm.

19. The method of any one of claims 1-18, wherein the implant releases about 0.1 mg to about 10 mg of the kappa-opioid receptor agonist per day, about 0.1 mg to about 0.5 mg of the kappa-opioid receptor agonist per day, about 0.5 mg to about 1.0 mg of the kappa-opioid receptor agonist per day, about 1.0 mg to about 3.0 mg of the kappa-opioid receptor agonist per day, or about 3.0 mg to about 5.0 mg of the kappa-opioid receptor agonist per day.

20. The method of any one of claims 1-19, wherein the implant is administered subcutaneously.

21. The method of any one of claims 1-20, wherein the implant comprises a core comprising the sustained release composition and a first layer comprising a first-layer biocompatible polymeric matrix surrounding the core.

22. The method of claim 21, wherein the first-layer biocompatible polymeric matrix comprises ethylene vinyl acetate (EVA) copolymer, crosslinked poly(vinyl alcohol), poly(hydroxy ethylmethacrylate), an acyl substituted cellulose acetate, a hydrolyzed alkylene-vinyl acetate copolymer, polyvinyl chloride, polyvinyl acetate, a polyvinyl alkyl ether, polyvinyl fluoride, polycarbonate, polyurethane, polyamide, polysulphone, styrene acrylonitrile copolymer, crosslinked poly(ethylene oxide), a poly(alkylene), poly(vinyl imidazole), a poly(ester), poly(ethylene terephthalate), polyphosphazene, a chlorosulphonated polyolefin, poly-lactide (PLA), poly-glycolide (PGA), poly(lactic-co-glycolic acid) (PLGA), or combinations thereof.

23. The method of claim 21, wherein the first-layer biocompatible polymeric matrix comprises ethylene vinyl acetate (EVA) copolymer.

24. The method of claim 23, wherein the EVA copolymer of the first-layer biocompatible polymeric matrix comprises about 20% to about 40% vinyl acetate of the total weight of the copolymer.

25. The method of claim 23, wherein the EVA copolymer of the first-layer biocompatible polymeric matrix comprises about 33% vinyl acetate of the total weight of the copolymer.

26. The method of any one of claims 21-25, wherein the implant further comprises one or more additional layers comprising a biocompatible polymeric matrix.

27. The method of any one of claims 21-26, wherein any of the core, the first layer, and the one or more additional layers if present, further comprise one or more additional pharmaceutical substances.

28. The method of any one of claims 1-27, wherein the implant remains in the individual for at least about 3 months, at least about 6 months, at least about 9 months, at least about 12 months, at least about 15 months, at least about 18 months, at least about 21 months, or at least about 24 months.

29. The method of claim 28, wherein the concentration of pharmaceutical substance in the blood plasma is approximately constant or essentially constant for at least about 3 months, at least about 6 months, at least about 9 months, at least about 12 months, at least about 15 months, at least about 18 months, at least about 21 months or at least about 24 months.

30. An implant comprising a sustained release composition, wherein the sustained release composition comprises a kappa-opioid receptor agonist and a biocompatible polymeric matrix,
wherein the implant comprises a core comprising the sustained release composition and a first layer comprising a first-layer biocompatible polymeric matrix surrounding the core; and
wherein the sustained release composition is configured to release a therapeutically effective amount of the kappa-opioid receptor agonist after administration.

31. The implant of claim 30, wherein the implant further comprises one or more additional layers comprising a biocompatible polymeric matrix.

32. The implant of claim 30 or claim 31, wherein any of the core, the first layer, and the one or more additional layers if present, further comprise one or more additional pharmaceutical substances.

33. The method of any one of claims 1-29, wherein the pruritus is chronic pruritus.

34. The method of any one of claims 1-29, wherein the pruritus is uremic pruritus.

35. The method of any one of claims 1-29, wherein the pruritus arises from dermatitis.

36. The method of any one of claims 1-29, wherein the pruritus arises from atopic dermatitis.

37. The method of any one of claims 1-29, wherein the pruritus arises from chronic liver disease.

38. The method of any one of claims 1-29, wherein the pruritus arises from notalgia paresthetica.
